(19) United States
(12) Patent Application Publication
(10) Pub. No.: US 2024/0082156 A1
(43) Pub. Date: Mar. 14, 2024

(54) METHOD FOR PRODUCING LIPOSOMES ENTRAPPING CYCLOSPORINE A

(71) Applicants: Mahmoud Reza Jaafari, Mashhad (IR); Seyed Ali Nazeri, Tehran (IR); Seyed Mahdi Rezayat, Tehran (IR); Ali Reza Partoazar, Tehran (IR)

(72) Inventors: Mahmoud Reza Jaafari, Mashhad (IR); Seyed Ali Nazeri, Tehran (IR); Seyed Mahdi Rezayat, Tehran (IR); Ali Reza Partoazar, Tehran (IR)

(73) Assignees: Mahmoud Reza Jaafari, Mashhad (IR); Seyed Ali Nazeri, Tehran (IR); Seyed Mahdi Rezayat, Tehran (IR); Ali Reza Partoazar, Tehran (IR)

(21) Appl. No.: 18/509,447

(22) Filed: Nov. 15, 2023

Related U.S. Application Data
(63) Continuation-in-part of application No. PCT/IB2022/054573, filed on May 17, 2022.
(60) Provisional application No. 63/189,229, filed on May 17, 2021.

(51) Int. Cl. A61K 9/127; A61P 37/06

(52) U.S. Cl. CPC A61K 9/1271; A61K 9/1277; A61P 37/06

(57) ABSTRACT

A method for producing liposomes entrapping Cyclosporine A (CsA). The method comprising preparing a suspension of CsA-free liposomes with a pH level of 7, preparing a mixture with a pH level of 7 by adding the suspension of CsA-free liposomes in the sucrose phosphate buffer to an ethanolic solution of CsA, and forming the liposomes entrapping CsA by incubating the mixture at a temperature level between 42° C. and 48° C. for a time duration between 55 and 65 minutes.

### CROSS-REFERENCE TO RELATED APPLICATION

This application is a continuation-in-part of PCT Application PCT/IB2022/054573, filed on May 17, 2022, entitled “METHOD FOR PRODUCING LIPOSOMES ENTRAPPING CYCLOSPORINE A” which claims the benefit of priority from U.S. Provisional Patent Application Ser. No. 63/189,229, filed on May 17, 2021, entitled “NANOLIPOSOME FORMULATION OF CYCLOSPORINE A” which are both incorporated herein by reference in their entirety.

### TECHNICAL FIELD

The present disclosure generally relates to an exemplary method for producing exemplary liposomes entrapping Cyclosporine A (CsA), and in particular to an exemplary method for loading CsA into exemplary liposomes with a high encapsulation efficiency.

### BACKGROUND

Organ transplantation has become a potential therapy option for patients with end-stage organ failure thanks to the discovery of immunosuppressive medicines. Despite the approval of a variety of immunosuppressive drugs over the previous two decades, long-term outcomes for allografts have remained unchanged and patients continue to face a variety of side effects. Cyclosporine A (CsA) is one of these medicines which may be commonly utilized to prevent transplanted organ rejection. CsA may have a poor pharmacokinetic profile, insufficient delivery to target immunomodulatory tissues, and serious off-target adverse effects. In this context, new formulation approaches, such as liposomes, appear to hold considerable promise for improved CsA delivery and therapeutic efficiency.

Liposomal drug formulations consist of vesicular lipid structures that may be commonly used to improve delivery of both hydrophilic and hydrophobic drugs to their target tissues. By using a liposomal formulation, a drug may potentially provide better targeted delivery over an extended period. There are two primary methods for creating pharmaceutical liposome formulations: passive and active methods. In passive method, drugs may be combined with lipids before formation of vesicles. This method may include dissolving dried lipid films in an aqueous solution containing a drug of interest, however, it may be limited to water-soluble (hydrophilic) drugs and may have a low encapsulation efficiency. On the other hand, active loading includes loading drugs into lipid vesicles after they have been produced. This technique may be highly efficient, resulting in increased intra-liposomal drug concentrations and minimal drug waste.

“pH gradient” method is a widely used active method for loading drug molecules into liposomes, which is typically achieved by establishing a transmembrane pH gradient. Given that CsA is a hydrophobic drug with low water solubility (approximately 27 μg/mL), it may not be effectively loaded into liposomes using passive method. Conversely, employing active techniques like pH gradient method for loading CsA into liposomes has proven to be inefficient, resulting in significantly low amounts of trapped CsA within liposomes. Furthermore, a suspension of CsA-containing liposomes produced by conventional passive and active methods may lack stability and may potentially precipitate over time. Therefore, there is need to develop an effective method that may result in a high encapsulation efficiency of CsA into liposomes and generate a more stable CsA liposomal formulation in a pharmaceutical dosage form.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more exemplary aspects in a simplified form as a prelude to the more detailed description that is presented later. The proper scope of the present disclosure may be ascertained from the claims set forth below in view of the detailed description below and the drawings.

One or more exemplary embodiments describe an exemplary method for producing liposomes entrapping Cyclosporine A (CsA). In an exemplary embodiment, an exemplary method may include preparing an exemplary suspension of CsA-free liposomes in an exemplary sucrose phosphate buffer. In an exemplary embodiment, an exemplary suspension of CsA-free liposomes in an exemplary sucrose phosphate buffer may have a pH level of about 7 and may comprises CsA-free liposomes with a concentration between about 45 mM and 55 mM by volume of an exemplary suspension of CsA-free liposomes in an exemplary sucrose phosphate buffer. In an exemplary embodiment, preparing an exemplary suspension of CsA-free liposomes in an exemplary sucrose phosphate buffer may comprise preparing a lipid solution by dissolving an exemplary plurality of lipids and alpha tocopherol in chloroform; forming a lipid film by evaporating chloroform from an exemplary lipid solution; and preparing an exemplary suspension of vesicles by hydrating an exemplary lipid film with an exemplary mannitol acetate buffer.

In an exemplary embodiment, preparing an exemplary suspension of vesicles by hydrating an exemplary lipid film with an exemplary mannitol acetate buffer may comprise preparing a mixture of an exemplary lipid film and an exemplary mannitol acetate buffer by adding an exemplary mannitol acetate buffer with a pH level of 7 and a temperature level between about 42° C. and 48° C. to an exemplary lipid film. In an exemplary embodiment, an exemplary mannitol acetate buffer may have a mannitol concentration between about 295 mM and 310 mM by volume of an exemplary mannitol acetate buffer. In an exemplary embodiment, preparing an exemplary suspension of vesicles by hydrating an exemplary lipid film with an exemplary mannitol acetate buffer may further comprise forming an exemplary suspension of vesicles by agitating an exemplary mixture of an exemplary lipid film and an exemplary mannitol acetate buffer while a temperature level of an exemplary mixture of an exemplary lipid film with an exemplary mannitol acetate buffer may be maintained between about 42° C. and 48° C.

In an exemplary embodiment, preparing an exemplary suspension of CsA-free liposomes in an exemplary sucrose phosphate buffer may further comprise extruding an exemplary suspension of vesicles through an exemplary polycarbonate membrane with a predetermined pore size; and forming an exemplary suspension of CsA-free liposomes in an exemplary sucrose phosphate buffer by dialyzing an exemplary extruded suspension of vesicles against an exemplary sucrose phosphate buffer with a pH level of about 7 and a sucrose concentration between about 270 mM and 290 mM.

In an exemplary embodiment, an exemplary method may further comprise preparing an exemplary mixture with a pH level of about 7 by adding an exemplary suspension of CsA-free liposomes in an exemplary sucrose phosphate buffer to an exemplary ethanolic solution of CsA with a CsA concentration between about 70 mM and 90 mM; and forming exemplary liposomes entrapping CsA by incubating an exemplary mixture at a temperature level between about 42° C. and 48° C. for a time duration between about 55 and 65 minutes.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of one or more exemplary embodiments. One or more exemplary embodiments will now be described by way of example in association with the accompanying drawings in which:

FIG. 1A illustrates an exemplary method for producing exemplary Cyclosporine A (CsA)-entrapping liposomes, consistent with one or more exemplary embodiments of the present disclosure;

FIG. 1B illustrates an exemplary method for preparing an exemplary suspension of CsA-free liposomes in an exemplary sucrose phosphate buffer, consistent with one or more exemplary embodiments of the present disclosure;

FIG. 1C illustrates an exemplary method for hydrating an exemplary lipid film with an exemplary mannitol acetate buffer with a pH level of about 7, consistent with one or more exemplary embodiments of the present disclosure;

FIG. 2 illustrates exemplary transmission electron microscope (TEM) images of exemplary CsA-entrapping liposomes, consistent with one or more exemplary embodiments of the present disclosure;

FIG. 3 illustrates a bar chart representing in vitro uptake of an exemplary free CsA and exemplary CsA-entrapping liposomes into exemplary human primary T cells at 37° C., consistent with one or more exemplary embodiments of the present disclosure;

FIG. 4 illustrates a bar chart representing in vitro uptake of an exemplary free CsA and exemplary CsA-entrapping liposomes into exemplary human primary T cells at 4° C., consistent with one or more exemplary embodiments of the present disclosure;

FIG. 5 illustrates a bar chart representing carboxyfluorescein succinimidyl ester (CFSE) fluorescence intensity of T cells following a 4-day incubation, at 37° C. and 5% CO₂, with exemplary samples of free CsA, CsA-entrapping liposomes, and CsA-free liposomes, consistent with one or more exemplary embodiments of the present disclosure;

FIG. 6 illustrates a bar chart representing immunosuppressive activity of exemplary free CsA, CsA-entrapping liposomes, and CsA-free liposomes against an exemplary sheep red blood cells (SRBC)-induced delayed-type hypersensitivity (DTH) reaction in rats, determined by measuring inflammatory edema of rats' left hind footpad, 24 h after subcutaneous injection of SRBCs, consistent with one or more exemplary embodiments of the present disclosure;

FIG. 7 illustrates a bar chart representing immunosuppressive activity of exemplary free CsA, CsA-entrapping liposomes, and CsA-free liposomes against an exemplary SRBC-induced DTH reaction in rats, determined by measuring an exemplary serum level of Interleukin-2 (IL-2) fourteen days after subcutaneous injection of SRBC, consistent with one or more exemplary embodiments of the present disclosure;

FIG. 8 illustrates a bar chart representing immunosuppressive activity of exemplary free CsA, CsA-entrapping liposomes, and CsA-free liposomes against an exemplary SRBC-induced DTH reaction in rats, determined by measuring serum levels of IgM fourteen days after subcutaneous injection of SRBC, consistent with one or more exemplary embodiments of the present disclosure;

FIG. 9 illustrates a bar chart representing biodistribution profile of both exemplary free CsA and CsA-entrapping liposomes, determined by measuring serum level of CsA at various time points following intravenous injection of exemplary CsA-entrapping liposomes and free CsA in rats, consistent with one or more exemplary embodiments of the present disclosure;

FIG. 10 illustrates a bar chart representing biodistribution profile of both exemplary free CsA and CsA-entrapping liposomes, determined by measuring levels of extracted CsA from various tissues 48 h after intravenous administration of exemplary CsA-entrapping liposomes and free CsA in rats, consistent with one or more exemplary embodiments of the present disclosure; and

FIG. 11 illustrates microscopic images representing histopathological features of exemplary transplanted skin tissues in exemplary host mice treated with a single dose (2 mg/mL) of exemplary free CsA, CsA-entrapping liposomes, and CsA-free liposomes, consistent with one or more exemplary embodiments of the present disclosure.

### DETAILED DESCRIPTION

In the following detailed description, numerous specific details are set forth by way of examples to provide a thorough understanding of the relevant teachings related to exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments. For purposes of explanation, specific nomenclature is set forth to provide a thorough understanding of one or more exemplary embodiments. However, it will be apparent to one skilled in the art that these specific details are not required to practice the disclosed exemplary embodiments. Descriptions of specific exemplary embodiments are provided only as representative examples. Various modifications to exemplary implementations will be plain to one skilled in the art, and the general principles defined herein may be applied to other implementations and applications without departing from the scope of one or more exemplary embodiments. The present disclosure is not intended to be limited to the implementations shown, but is to be accorded the widest possible scope consistent with the principles and features disclosed herein.

Cyclosporine A (CsA) is among the most effective immunosuppressive drugs used to prevent allograft rejection following organ transplantation. Despite the medical importance of CsA, several drawbacks have been identified with some of the existing CsA dosage forms. These drawbacks include, but are not limited to, considerable variability in CsA absorption and/or poor absorption of CsA after oral administration of CsA oily solution. Employing liposomes as a drug delivery system for CsA may result in enhanced CsA delivery and therapeutic effectiveness. However, it may be appreciated that employing conventional passive and active techniques to create CsA-containing liposomes may yield low encapsulation efficiency and reduced stability. Provided herein is an exemplary method for producing exemplary liposomes entrapping CsA. Exemplary liposomes entrapping CsA (also referred to as exemplary CsA-entrapping liposomes) may be used to prevent organ rejection after transplantation. “Cyclosporine A (CsA),” with the molecular formula C₆₂H₁₁₁N₁₁O₁₂, may be known as a potent immunosuppressive and/or a calcineurin inhibitor which may be used to prevent cellular rejection following solid organ transplantation. Possible synonyms for CsA may include, but are not limited to, Cyclosporine, Cyclosporin, and/or Cyclosporin A.

“Liposomes” may comprise bilayer vesicles commonly employed as a delivery system and/or carrier for transporting drugs to target tissues and/or organs. Therefore, liposomes may reduce systemic adverse effects of drugs in other organs and/or tissues. Liposomes may be made of phospholipids with a polar end and a nonpolar chain that may self-assemble into bilayer vesicles, with exemplary polar ends facing towards an exemplary aqueous medium/interior space of an exemplary bilayer vesicle and exemplary nonpolar ends creating an exemplary bilayer around exemplary aqueous medium/interior space.

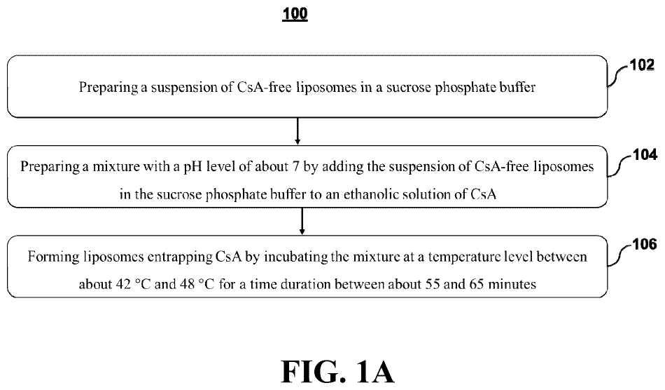

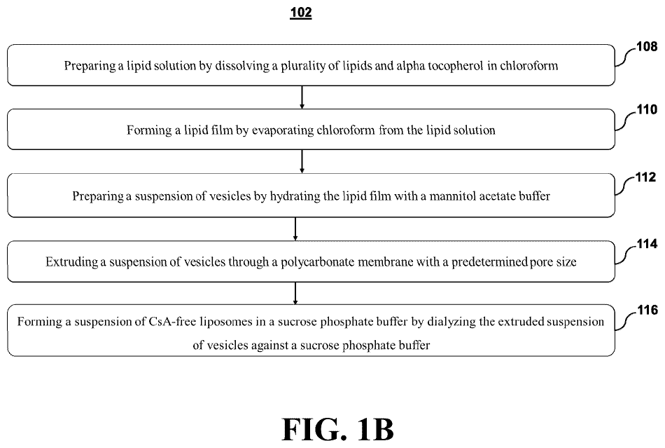

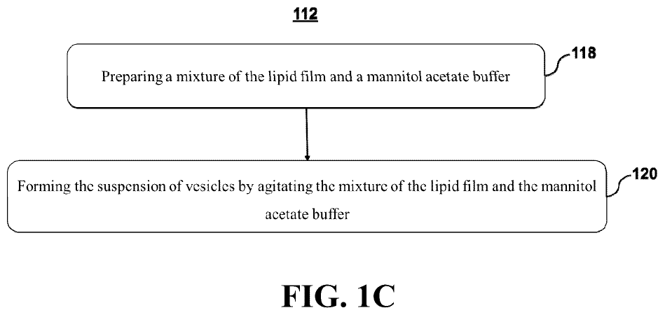

An exemplary embodiment may be directed to an exemplary method for producing exemplary liposomes entrapping CsA (i.e., exemplary CsA-entrapping liposomes). FIG. 1A illustrates exemplary method 100 for producing exemplary CsA-entrapping liposomes, consistent with one or more exemplary embodiments of the present disclosure. In an exemplary embodiment, exemplary method 100 may include: preparing an exemplary suspension of CsA-free liposomes in an exemplary sucrose phosphate buffer (step 102); preparing an exemplary mixture with a pH level of about 7 by adding an exemplary suspension of CsA-free liposomes in an exemplary sucrose phosphate buffer to an exemplary ethanolic solution of CsA (step 104); and forming exemplary liposomes entrapping CsA by incubating an exemplary mixture at a temperature level between about 42° C. and 48° C. for a time duration between about 55 and 65 minutes (step 106).

In further detail with respect to step 102, step 102 may include preparing an exemplary suspension of CsA-free liposomes in an exemplary sucrose phosphate buffer. In an exemplary embodiment, an exemplary suspension of CsA-free liposomes in an exemplary sucrose phosphate buffer may have a pH level of about 7 and may comprise exemplary CsA-free liposomes with a final concentration of about 45-55 mM (with respect to an exemplary final volume of an exemplary suspension of CsA-free liposomes in an exemplary sucrose phosphate buffer). Each of exemplary CsA-free liposomes may comprise an exemplary lipid bilayer structure with a closed concentric lamella, in which an exemplary closed concentric lamella may enclose one or more exemplary aqueous-containing compartments. Exemplary CsA-free liposomes may include one or more exemplary phospholipids, but other molecules having similar dimensions and molecular shape to exemplary phospholipids (e.g., having an exemplary hydrophobic moiety and an exemplary hydrophilic moiety) may also be used for forming an exemplary lipid bilayer of exemplary CsA-free liposomes. Exemplary phospholipids that may be used for preparing exemplary CsA-free liposomes may have an exemplary hydrophobic tail and an exemplary hydrophilic head. In an exemplary embodiment, exemplary lipids may include naturally occurring and/or synthetic lipid compounds. In an exemplary embodiment, exemplary CsA-free liposomes may be cationic, neutral, or anionic depending on exemplary type of hydrophilic group. For instance, when a compound with a sulphate or a phosphate group is used, exemplary produced CsA-free liposomes may be anionic. In an exemplary embodiment, when amino-containing lipids are used, exemplary produced CsA-free liposomes may be cationic. In an exemplary embodiment, exemplary lipid bilayer of each exemplary CsA-free liposome may comprise a plurality of exemplary lipids and alpha tocopherol. In an exemplary embodiment, exemplary lipids may comprise a plurality of exemplary phospholipids, including at least one of dioleoyl phosphatidylethanolamine, dioleoyl phosphatidylserine, an exemplary polyethylene glycol (PEG)-modified phospholipid, and a combination thereof. In an exemplary embodiment, exemplary lipids may further include a plurality of exemplary cholesterol molecules dispersed among exemplary phospholipids. In an exemplary embodiment, an exemplary PEG-modified phospholipid may be a 1,2-Distearoyl-sn-glycero-3-phosphoethanolamine-PEG conjugate.

In an exemplary embodiment, details of step 102 for preparing an exemplary suspension of CsA-free liposomes in an exemplary sucrose phosphate buffer are described in context of elements presented in FIG. 1B. FIG. 1B illustrates an exemplary method of step 102 for preparing an exemplary suspension of CsA-free liposomes in an exemplary sucrose phosphate buffer, consistent with one or more exemplary embodiments of the present disclosure. In an exemplary embodiment, an exemplary method of step 102 may comprise: preparing an exemplary lipid solution by dissolving a plurality of exemplary lipids and alpha tocopherol in chloroform (step 108); forming an exemplary lipid film by evaporating chloroform from an exemplary lipid solution (step 110); preparing an exemplary suspension of vesicles by hydrating an exemplary lipid film with an exemplary mannitol acetate buffer (step 112); extruding an exemplary suspension of vesicles through an exemplary polycarbonate membrane with a predetermined pore size (step 114); and forming an exemplary suspension of CsA-free liposomes in an exemplary sucrose phosphate buffer by dialyzing an exemplary extruded suspension of vesicles against an exemplary sucrose phosphate buffer (step 116).

In further detail with respect to step 108, step 108 may include preparing an exemplary lipid solution by dissolving a plurality of exemplary lipids and alpha tocopherol in chloroform. In an exemplary embodiment, preparing an exemplary lipid solution by dissolving a plurality of exemplary lipids and alpha tocopherol in chloroform may include preparing a chloroform-lipid solution by dissolving dioleoyl phosphatidylethanolamine (e.g., in form of powder), dioleoyl phosphatidylserine (e.g., in form of powder), 1,2-distearoyl-sn-glycero-3-phosphoethanolamine-N-[amino(polyethyleneglycol)-2000] (e.g., in form of powder), cholesterol (e.g., in form of powder), and alpha tocopherol (e.g., in form of liquid) in chloroform, in an exemplary laboratory container. An exemplary laboratory container may include, but is not limited to, beakers, tins, flasks, bottles, buckets, basins, bowls, vials, tubes, barrels, cannisters, etc. In an exemplary embodiment, dissolving a plurality of exemplary lipids and alpha tocopherol in chloroform may include dissolving dioleoyl phosphatidylethanolamine with a final concentration of about 15-19 mM (by volume of an exemplary chloroform-lipid solution), dioleoyl phosphatidylserine with a final concentration of about 18-22 mM (by volume of an exemplary chloroform-lipid solution), 1,2-distearoyl-sn-glycero-3-phosphoethanolamine-N-[amino(polyethyleneglycol)-2000] with a final concentration of about 1-3.5 mM (by volume of an exemplary chloroform-lipid solution), cholesterol with a final concentration of about 7.5-12.5 mM (by volume of an exemplary chloroform-lipid solution), and alpha tocopherol with a final concentration of about 0.1-0.9 mM (by volume of an exemplary chloroform-lipid solution) in chloroform (e.g., 500 mL chloroform in an exemplary flask) while stirring, e.g., using a magnetic stirrer (with a speed between about 300 rpm and 2000 rpm).

Furthermore, in one or more exemplary embodiments, dissolving a plurality of exemplary lipids may comprise dissolving a plurality of lipid molecules selected from the group consisting of exemplary dialiphatic chain lipids, such as diglycerides, phospholipids, dialiphatic glycolipids, single lipids such as glycosphingolipid and sphingomyelin, steroids/sterols such as cholesterol and derivates of cholesterol, and a combination thereof in chloroform. Examples of phospholipids may include—but are not limited to—1,2-dimyristoyl-sn-glycero-3-phosphocholine (DMPC), 12-dilauroyl-sn-glycero-3-phosphocholine (DLPC), 1,2-dipalmitoyl-sn-glycero-3-phosphocholine (DPPC), 1-palmitoyl-2-oleoyl-sn-glycero-3-phosphatidylcholine (POPC), 1-palmitoyl-2-stearoyl-sn-glycero-3-phosphocholine (PSPC), 1,2-dioleoyl-sn-glycero-3-phosphocholine (DOPC), 1,2-di stearoyl-sn-gly cero-3-phosphocholine (DSPC), hydrogenated soy phosphatidylcholine (HSPC), 1,2-dipalmitoyl-sn-glycero-3-phospho-(1′-rac-glycerol) (sodium salt) (DPPG), 1,2-dimyristoyl-sn-glycero-3-phospho-(1′-rac-glycerol) (sodium salt) (DMPG), 1-palmitoyl-2-stearoyl-sn-glycero-3-phospho-(1′-rac-glycerol) (sodium salt) (PSPG), 1,2-dioleoyl-sn-glycero-3-phospho-(1′-rac-glycerol) (DOPG), 1,2-distearoyl-sn-glycero-3-phospho-(1′-rac-glycerol) (sodium salt) (DSPG), 1,2-di stearoyl-sn-gly cero-3-phospho-L-serine (sodium salt) (DSPS), 1,2-dimyristoyl-sn-glycero-3-phospho-L-seine (sodium salt) (DMPS), 1,2-dipalmitoyl-sn-glycero-3-phosphate (sodium salt) (DPPA), 1,2-dipalmitoyl-sn-glycero-3-phospho-L-serine (sodium salt) (DPPS), 1,2-dioleoyl-sn-glycero-3-phospho-L-serine (DOPS), 1,2-dimyristoyl-sn-glycero-3-phosphate (sodium salt) (DMPA), 1,2-dioleoyl-sn-glycero-3-phosphate (sodium salt) (DOPA), 1,2-distearoyl-sn-glycero-3-phosphate (sodium salt) (DSPA), 1,2-dioleoyl-sn-glycero-3-phosphoethanolamine (DOPE), 1,2-dipalmitoyl-9n-glycero-3-phosphoethanolamine (DPPE), 1-palmitoyl-2-oleoyl-sn-gly cero-3-phosphoethanolamine (POPE), 1,2-di stearoyl-sn-gly cero-3-phosphoinositol (ammonium salt) (DSPI), 1,2-distearoyl-sn-glycero-3-phosphoethanolamine (DSPE), 1,2-dioleoyl-sn-glycero-3-phospho-(1-myo-inositol) (ammonium salt) (DOPI), 1,2-dipalmitoyl-sn-glycero-3-phospho-(1-myo-inositol) (ammonium salt) (DPPI), L-α-phosphatidylcholine (EPC), cardiolipin, and L-α-phosphatidylethanolamine (EPE).

In further detail with respect to step 110, step 110 may include forming an exemplary lipid film by evaporating chloroform from an exemplary lipid solution. In an exemplary embodiment, “lipid film” may refer to a thin layer of lipids that may be formed by evaporating an exemplary solvent (e.g., chloroform) from an exemplary lipid solution. In an exemplary embodiment, evaporating chloroform from an exemplary lipid solution may include: placing an exemplary lipid solution in an exemplary container (e.g., a round-bottom flask or a test tube); connecting an exemplary container to an exemplary rotary evaporator; setting up an exemplary rotary evaporator, under reduced pressure (i.e., vacuum of about 200 mbar), at a predetermined temperature (e.g., 30-40° C.); and evaporating chloroform solvent, using an exemplary rotary evaporator, until all solvent is removed (e.g., after about 8-24 hours), leaving behind a thin, uniform layer of dried lipids (i.e., an exemplary lipid film) on inner walls of an exemplary container.

In further detail with respect to step 112, step 112 may include preparing an exemplary suspension of vesicles by hydrating an exemplary lipid film with an exemplary mannitol acetate buffer. “Hydration” may refer to a common step in process of liposome preparation, which may be carried out after formation and drying of a lipid film. In an exemplary hydration step, a dried lipid film may be hydrated by suspending it in an aqueous buffer solution while agitating at a temperature higher than a lipid's transition temperature. A hydration step may result in formation of a population of heterogeneous vesicles that may vary in size and shape, and may encapsulate components of aqueous buffer solution (e.g., a chemical or biological drug).

In an exemplary embodiment, details of step 112 for preparing an exemplary suspension of vesicles by hydrating an exemplary lipid film with an exemplary mannitol acetate buffer are described in context of elements presented in FIG. 1C. FIG. 1C illustrates an exemplary method of step 112 for hydrating an exemplary lipid film with an exemplary mannitol acetate buffer with a pH level of about 7, consistent with one or more exemplary embodiments of the present disclosure. In an exemplary embodiment, an exemplary method of step 112 may comprise: preparing an exemplary mixture of an exemplary lipid film and an exemplary mannitol acetate buffer (step 118); and forming an exemplary suspension of vesicles by agitating an exemplary mixture of an exemplary lipid film and an exemplary mannitol acetate buffer (step 120).

In further detail with respect to step 118, step 118 may include preparing an exemplary mixture of an exemplary lipid film and an exemplary mannitol acetate buffer. In an exemplary embodiment, preparing an exemplary mixture of an exemplary lipid film and an exemplary mannitol acetate buffer may include preparing an exemplary mixture of an exemplary lipid film and an exemplary mannitol acetate buffer by adding an exemplary mannitol acetate buffer (e.g., using a graduated pipette, volumetric pipette, burette, etc.) with a pH level of about 7 and a temperature level between about 42° C. and 48° C. (e.g., 45° C.) to an exemplary lipid film, in an exemplary laboratory container. In an exemplary embodiment, an exemplary mannitol acetate buffer may have a mannitol concentration between about 295 mM and 310 mM with respect to an exemplary final volume of an exemplary mannitol acetate buffer. In an exemplary embodiment, an exemplary mannitol acetate buffer may have a mannitol concentration between about 300 mM with respect to an exemplary final volume of an exemplary mannitol acetate buffer. In an exemplary embodiment, preparing about 1 L of an exemplary mannitol acetate buffer with a mannitol concentration of about 300 mM and a pH level of about 7 may include: weighing out approximately 54.65 g of mannitol and dissolving it in about 800 mL of distilled water using a beaker; using a pH meter and electrode to measure pH of an exemplary mannitol solution and adjusting pH by adding NaOH and acetic acid to an exemplary mannitol solution (while stirring using a magnetic stirrer) until reaching a pH of about 7; transferring an exemplary mannitol acetate buffer to a volumetric flask and diluting it up to about 1 L with distilled water.

In further detail with respect to step 120, step 120 may include forming an exemplary suspension of vesicles by agitating an exemplary mixture of an exemplary lipid film and an exemplary mannitol acetate buffer. In an exemplary embodiment, agitating an exemplary mixture of an exemplary lipid film and an exemplary mannitol acetate buffer may include swirling or vortexing an exemplary mixture of an exemplary lipid film and an exemplary mannitol acetate buffer for a time duration of at least 30 minutes, while a temperature level of an exemplary mixture of an exemplary lipid film with an exemplary mannitol acetate buffer is maintained between about 42° C. and 48° C. (e.g., 45° C.), using a magnetic stirrer or an agitation machine with a speed between about 60 to 1000 rpm. In an exemplary embodiment, forming an exemplary suspension of vesicles may include forming an exemplary suspension of multilamellar vesicles (MLVs). “Multilamellar vesicles (MLVs)” may be described as particles may have an aqueous inner compartment, separated from an outer environment/medium by a membrane that may consist of one or more bilayers, with each of these bilayers forming an individual vesicle.

With further reference to FIG. 1B, in an exemplary embodiment, step 114 may include extruding an exemplary suspension of vesicles through an exemplary polycarbonate membrane with a predetermined pore size. “Extrusion” may refer to a technique used to homogenize and reduce the size of liposomes, particularly multilamellar vesicles (MLVs), to create large unilamellar vesicles (LUVs) with a more uniform size distribution. Extrusion process may involve forcing a liposome suspension through a polycarbonate membrane with defined pore sizes under pressure. In an exemplary embodiment, extruding an exemplary suspension of vesicles through an exemplary polycarbonate membrane with a predetermined pore size may include extruding an exemplary suspension of vesicles using one or more exemplary lipid extruders, in which two syringes may be connected by a holder containing a polycarbonate membrane. In an exemplary embodiment, extruding an exemplary suspension of vesicles through an exemplary polycarbonate membrane with a predetermined pore size may include: i) preheating an exemplary suspension of vesicles to a temperature level of about 42-48° C.; ii) filling an exemplary first syringe of an exemplary lipid extruder with an exemplary preheated suspension of vesicles and connecting an exemplary filled syringe to an exemplary lipid extruder; iii) slowly pushing down on an exemplary first syringe's plunger while holding on the other syringe (i.e., an exemplary second syringe) firmly in place, and forcing an exemplary preheated suspension of vesicles through an exemplary polycarbonate membrane (with a pore size of about 200 nm) into the other syringe; iv) extruding an exemplary 200 nm-sized suspension of vesicles (by repeating exemplary steps (i) to (iii)) through an exemplary polycarbonate membrane with a pore size of about 100 nm; and v) extruding an exemplary 100 nm-sized suspension of vesicles (by repeating exemplary steps (i) to (iii)) through an exemplary polycarbonate membrane with a pore size of about 50 nm.

In further detail with respect to step 116, step 116 may include forming an exemplary suspension of CsA-free liposomes in an exemplary sucrose phosphate buffer by dialyzing an exemplary extruded suspension of vesicles against an exemplary sucrose phosphate buffer. In an exemplary embodiment, dialyzing an exemplary extruded suspension of vesicles against an exemplary sucrose phosphate buffer may include dialyzing an exemplary extruded suspension of vesicles against an exemplary sucrose phosphate buffer with a pH level of about 7 and a sucrose concentration between about 270 mM and 290 mM (with respect to an exemplary final volume of an exemplary sucrose phosphate buffer). In an exemplary embodiment, dialyzing an exemplary extruded suspension of vesicles against an exemplary sucrose phosphate buffer may include dialyzing an exemplary extruded suspension of vesicles against an exemplary sucrose phosphate buffer with a pH level of about 7, a sucrose concentration of about 280 mM, and a phosphate concentration of about 10 mM (with respect to an exemplary final volume of an exemplary sucrose phosphate buffer). “Dialysis” may refer to a purification technique used to separate molecules based on their size and/or charge. In the context of liposome preparation, dialysis may be employed to remove non-encapsulated solutes or residual impurities from a liposome suspension. In an exemplary embodiment, dialyzing an exemplary extruded suspension of vesicles against an exemplary sucrose phosphate buffer with a pH level of about 7 and a sucrose concentration between about 270 mM and 290 mM may include: selecting an exemplary semi-permeable membrane with a molecular weight cut-off (MWCO) of about 12-14 kDa that may allow the passage of smaller molecules (e.g., unencapsulated solutes, residual impurities) while retaining larger molecules (i.e., exemplary CsA-entrapping liposomes); preparing an exemplary dialysis tubing by cutting a certain length (e.g., 5 cm) of an exemplary dialysis tubing and closing one end by tying a knot or using an exemplary clamp; rinse an exemplary dialysis tubing in distilled water to remove any preservatives or contaminants; transferring an exemplary extruded suspension of vesicles into an exemplary open end of an exemplary dialysis tubing, making sure not to overfill an exemplary dialysis tubing as it may cause leaking or bursting during dialysis; closing other end of an exemplary tubing securely with a knot or clamp; placing an exemplary filled dialysis tubing into an exemplary laboratory container (e.g., a beaker) filled with an exemplary sucrose phosphate buffer with a pH level of about 7 and a sucrose concentration between about 270 mM and 290 mM (volume of an exemplary sucrose phosphate buffer may be at least 10-20 times greater than volume of an exemplary extruded suspension of vesicles to maximize removal of impurities); keeping an exemplary sucrose phosphate buffer by gently stirring during dialysis to maintain concentration gradients between an exemplary extruded suspension of vesicles and an exemplary sucrose phosphate buffer, which may facilitate removal of unwanted small molecules from an exemplary extruded suspension of vesicles; replacing part or all of an exemplary sucrose phosphate buffer with an exemplary fresh sucrose phosphate buffer, at regular time intervals (e.g., every 4-8 hours); retrieving an exemplary purified suspension of CsA-free liposomes (for example after an exemplary 24-hour dialysis process) by removing (e.g., using a sampler) an exemplary suspension of CsA-free liposomes from an exemplary dialysis tubing and transferring an exemplary suspension of CsA-free liposomes to a laboratory container (e.g., a tube).

With further reference to FIG. 1A, in an exemplary embodiment, step 104 may include preparing an exemplary mixture with a pH level of about 7 by adding an exemplary suspension of CsA-free liposomes in an exemplary sucrose phosphate buffer to an exemplary ethanolic solution of CsA. In an exemplary embodiment, adding an exemplary suspension of CsA-free liposomes in an exemplary sucrose phosphate buffer to an exemplary ethanolic solution of CsA may include adding (e.g., using graduated pipette, volumetric pipettes, burette, etc.) an exemplary suspension of CsA-free liposomes in an exemplary sucrose phosphate buffer (formed in step 116 set forth in FIG. 1B) to an exemplary ethanolic solution of CsA with a CsA concentration between about 70 mM and 90 mM (by an exemplary final volume of an exemplary ethanolic solution of CsA) in an exemplary laboratory container (e.g., flask, beaker, tube, etc.), while stirring at a temperature level of 20-25° C. (i.e., room temperature), using a magnetic stirrer or agitation machine that may be set up to a speed between about 300-600 rpm. In an exemplary embodiment, an exemplary mixture may comprise about 8.3 mM CsA and about 50 mM CsA-free liposomes.

In further detail with respect to step 106, step 106 may include forming exemplary liposomes entrapping CsA by incubating an exemplary mixture at a temperature level between about 42° C. and 48° C. for a time duration between about 55 and 65 minutes. In an exemplary embodiment, incubating an exemplary mixture at a temperature level between about 42° C. and 48° C. for a time duration between about 55 and 65 minutes may include placing an exemplary laboratory container (e.g., a tube) containing an exemplary mixture in an incubator or a thermostatic water bath that may be set up at a temperature level of about 42-48° C. (e.g., 45° C.), for a time duration of about 55-65 minutes (e.g., 60 minutes).

### EXAMPLES

Hereinafter, one or more exemplary embodiments will be described in further detail with reference to examples. It will be obvious to a person having ordinary skill in the art that these examples may be for illustrative purposes only and are not to be interpreted to limit the scope of one or more exemplary embodiments.

### Example 1: Producing Liposomes Entrapping Cyclosporine a (CsA)

In this example, exemplary CsA-entrapping liposomes were produced using an exemplary method similar to exemplary method 100. Table 1 below shows components of an exemplary procedure for producing exemplary CsA-entrapping liposomes and their corresponding concentrations with respect to an exemplary final volume of chloroform solvent.

TABLE 1
Components of an exemplary procedure for producing exemplary
CsA-entrapping liposomes and their corresponding concentrations
with respect to an exemplary final volume of chloroform
solvent, consistent with one or more exemplary embodiments
of the present disclosure.

| | Concentration |
|---|---|
| | in chloroform |
| Ingredients | solution (mM) |
| Dioleoyl phosphatidylethanolamine | 17.5 |
| Dioleoyl phosphatidylserine | 20 |
| Cholesterol | 9.75 |
| Alpha tocopherol | 0.25 |
| 1,2-distearoyl-sn-glycero-3-phosphoethanolamine-N- | 2.5 |
| [amino(polyethyleneglycol)-2000] | |
| Cyclosporine A | 8.3 |

In brief, exemplary lipids and alpha tocopherol were dissolved in chloroform with final concentrations as set forth in Table 1 while vigorously agitating in an exemplary round-bottom flask. Then, chloroform was completely evaporated from an exemplary prepared lipid solution using an exemplary rotary evaporator, set up under vacuum at 40° C., to form a thin lipid film on inner walls of an exemplary round-bottom flask. Next, an exemplary suspension of multilamellar vesicle (MLVs) was produced by hydrating an exemplary dried lipid film in an exemplary mannitol acetate buffer (with a mannitol concentration of about 300 mM, pH=7) at a temperature level of about 20-25° C. (i.e., room temperature). An exemplary lipid film was agitated (using a magnetic stirrer) in mannitol acetate buffer, at a temperature level of about 45° C. for a time duration of about 30-60 minutes, to form an exemplary suspension of vesicles. To downsize exemplary formed vesicles, an exemplary suspension of vesicles was extruded through exemplary polycarbonate membranes with pore sizes of about 0.2 μm, 0.1 μm, and 0.05 μm, respectively. Next, an exemplary extruded suspension of vesicles was dialyzed against an exemplary sucrose phosphate buffer (9.5% (w/v) sucrose, 10 mM phosphate, pH=7) using an exemplary dialysis membrane to form an exemplary purified suspension of CsA-free liposomes. An exemplary suspension of CsA-free liposomes was then mixed with an exemplary ethanolic solution of CsA (having a CsA concentration of about 8.3 mM) with a volume ratio (CsA:liposome) of about 1:9, and incubated (in an incubator) at about 45° C. for a time duration of about 1 hour. To further eliminate residual impurities from an exemplary mixture, an exemplary mixture was dialyzed against an exemplary sucrose phosphate buffer at about 20-25° C. for a time duration of about 24 hours. Following an exemplary dialysis phase, an exemplary suspension of CsA-entrapping liposomes was obtained and collected in a sterile vial after filtering with a 0.22 vim polypropylene syringe filter.

### Example 2: Encapsulation Efficiency of CsA-Entrapping Liposomes

In this example, encapsulation efficiency (EE) of CsA in liposomes was examined using reverse-phase high-performance liquid chromatography (HPLC) before and after dialysis of exemplary CsA-entrapping liposomes. To this end, two samples (about 2 μL) including an exemplary pre-dialysis sample and an exemplary post-dialysis sample of exemplary CsA-entrapping liposomes were collected and dissolved in about 2 mL of absolute methanol (about 100% (v/v)). Then, about 40 μL of exemplary samples was injected into an exemplary HPLC column. An exemplary HPLC was conducted using an isocratic elution of 90% methanol and 10% water for a duration of about 10 minutes. HPLC flow rate was maintained at about 1 mL/minute, and CsA concentration was determined at a wavelength of about 205 nm. Encapsulation efficiency of CsA in liposomes was calculated using the following Equation (1):

$$\% EE = \frac{Amount of CsA After Purification}{Total Amount of Added CsA} \times 100 \qquad ( 1 )$$

Using Equation (1), encapsulation efficiency of an exemplary method for producing exemplary CsA-entrapping liposomes was measured to be about 61%. To compare an exemplary method described in “Example 1” with pH gradient method, exemplary ingredients set forth in Table 1 were dissolved in chloroform, then chloroform was removed with a rotary evaporator. Resultant film was then hydrated by mannitol acetate buffer (300 mM, pH=3.0) at about 20-25° C., and an exemplary suspension was passed through exemplary 200, 100, and 50 nm polycarbonate membranes using a thermobarrel mini extruder.

Next, an exemplary extruded liposome suspension was dialyzed three times in sucrose phosphate buffer solution (9.5% (w/v) sucrose, 10 mM phosphate, pH=7.0) through a dialysis membrane with 12-14 kDa molecular weight cut-off value. Afterwards, an ethanolic solution of CsA with a CsA concentration of about 8.3 mM was mixed with an exemplary CsA-free liposome suspension with a volume ratio (CsA:liposome) of 1:9 and incubated at 45° C. for about an hour. An exemplary mixture was then cooled to about 20-25° C. and dialyzed against an exemplary sucrose phosphate buffer for about 24 hours (three times) at about 20-25° C. (i.e., room temperature). Finally, an exemplary mixture was filtered through a 0.22 pin syringe filter and added into a sterile vial. An exemplary pH gradient method yielded an EE value of about 5.6%. Thus, it was found that EE value of an exemplary method set forth in “Example 1” was significantly greater than that of pH gradient method.

### Example 3: Morphology of CsA-Entrapping Liposomes

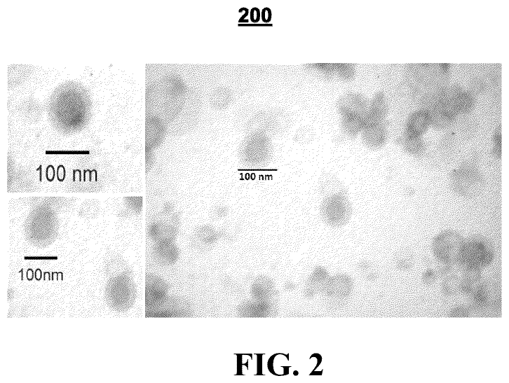

In this example, a transmission electron microscope (TEM) was utilized to examine morphological characteristics of exemplary CsA-entrapping liposomes at a voltage of about 120 kV. To prepare an exemplary sample for analysis, a solution containing these liposomes was diluted ten times in 2 mL of phosphate-buffered saline (PBS, 300 mM, pH=7.4). The diluted mixture was then placed on an exemplary carbon-coated copper grid for examination. Next, an exemplary grid was negatively stained with filtered uranyl acetate (2% (w/v), pH=7). Exemplary stained samples were allowed to air-dry before being imaged using TEM. FIG. 2 illustrates TEM images 200 of exemplary CsA-entrapping liposomes, consistent with one or more exemplary embodiments of the present disclosure. As shown in TEM images 200, exemplary CsA-entrapping liposomes were spherical and had an approximate diameter of 100 nm, which was slightly smaller than the measurement obtained through dynamic light scattering (DLS). Meanwhile, within the inner aqueous phase of exemplary CsA-encapsulating liposomes, CsA was observed as a compound alongside mannitol.

### Example 4: Physicochemical Properties of CsA-Entrapping Liposomes

In this example, long-term stability of exemplary CsA-entrapping liposomes was evaluated by determining z-average, polydispersity index (PDI), and zeta (0 potential after 0, 1, 3, 6, 12, and 18 months of storage at about 4° C. To measure z-average and polydispersity index, an exemplary suspension of CsA-entrapping liposomes was diluted in dextrose solution (5% w/v), and to measure z-potential, an exemplary suspension of CsA-entrapping liposomes was diluted in 3-(N-morpholino) propane sulfonic acid (MOPS) buffer (pH=7). Leakage of CsA from exemplary CsA-entrapping liposomes was measured using HPLC to assess chemical stability of exemplary produced CsA-entrapping liposomes. Table 2 below shows physicochemical properties of exemplary CsA-entrapping liposomes.

TABLE 2
Physicochemical properties of exemplary CsA-entrapping
liposomes, consistent with one or more exemplary
embodiments of the present disclosure.

| | Polydispersity | | |
|---|---|---|---|
| Zeta-Average | Index | Zeta potential | CsA Concentration |
| (nm) | (PDI) | (mV) | (mg/ml) |
| 143 ± 67 | 0.12 | −17 ± 7 | 6.3 ± 1.5 |

Table 3 presented below illustrates long-term physicochemical stability of exemplary CsA-encapsulating liposomes during storage in a refrigerator (i.e., 4° C.) for 18 months. As indicated by Table 3, exemplary CsA-encapsulating liposomes maintained their physical characteristics after long-term preservation in refrigerator. However, their CsA content progressively diminished by approximately 50% over 18 months of storage. Throughout this 18-month period, liposome size (Z-average), size distribution (polydispersity index or PDI), and surface charge (Z-potential) remained stable.

TABLE 3
Long-term physicochemical stability of exemplary CsA-entrapping
liposomes (during 18-month storage in refrigerator), consistent
with one or more exemplary embodiments of the present disclosure.

| | | Polydispersity | | CsA |
|---|---|---|---|---|
| | Z-Average | Index | Zeta Potential | Concentration |
| Months | (nm) | (PDI) | (mV) | (mg/ml) |
| 0 | 143 ± 67 | 0.120 | −17 ± 6 | 6.3 |
| 1 | 143 ± 67 | 0.086 | −18 ± 8 | 6.1 |
| 3 | 145 ± 61 | 0.120 | −19 ± 8 | 5.4 |
| 6 | 143 ± 55 | 0.112 | −19 ± 8 | 5.1 |
| 12 | 144 ± 65 | 0.120 | −21 ± 8 | 4 |
| 18 | 176 ± 66 | 0.23 | −26 ± 14 | 3.1 |

### Example 5: Evaluating In Vitro Cellular Uptake of CsA-Entrapping Liposomes

In this example, cellular uptake of exemplary CsA-entrapping liposomes was assessed (using HPLC method) in isolated human T-cells at 37° C. and 4° C. For this purpose, CsA-entrapping liposomes and free CsA (non-liposomal) were combined with T-cells (5.0×10⁴ cells) in 0.2 mL of Roswell Park Memorial Institute (RPMI) culture media, followed by a one-hour incubation at both 37° C. and 4° C. After washing twice with cold PBS and centrifuging (1800 g, 5 minutes), supernatant was removed, and cells were lysed using a solution containing 0.2 mL ZnSO₄ (10% w/v in water) and 0.4 mL acetonitrile. Lastly, HPLC method was employed to determine the amount of CsA present in sample solutions. Cellular uptake of CsA was calculated using the following Equation (1):

$$Cellular update of CsA = \frac{The amount of drug absorbed by the cells}{Total amount CsA added} \times 100 \qquad ( 2 )$$

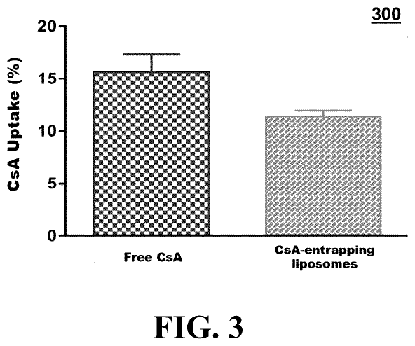

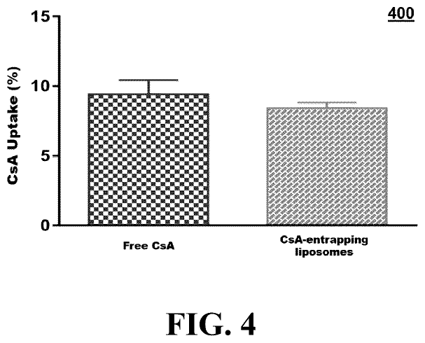

FIG. 3 illustrates bar chart 300 representing in vitro uptake of an exemplary free CsA and exemplary CsA-entrapping liposomes into exemplary human primary T cells at 37° C., consistent with one or more exemplary embodiments of the present disclosure. FIG. 4 illustrates bar chart 400 representing in vitro uptake of an exemplary free CsA and exemplary CsA-entrapping liposomes into exemplary human primary T cells at 4° C., consistent with one or more exemplary embodiments of the present disclosure. In further detail with respect to bar chart 300 and bar chart 400, when cells were exposed to both exemplary free CsA and CsA-entrapping liposomes at 4° C., the level of CsA uptake by T cells was identical. However, when T cells were treated at 37° C., there was a significant difference in CsA uptake between exemplary cells treated with free CsA and exemplary cells treated with CsA-entrapping liposomes. Compared to exemplary cells treated with free CsA, lower amounts of CsA were observed in cells treated with CsA-entrapping liposomes

### Example 6: Evaluating In Vitro Immunosuppressive Activity of CsA-Entrapping Liposomes

In this example, an exemplary fluorescent dye dilution method was employed to investigate in vitro immunosuppressive effects of exemplary CsA-entrapping liposomes and free CsA. In an exemplary embodiment, T cells were initially labelled in the dark using 10 μL of carboxyfluorescein succinimidyl ester (CFSE). Subsequently, exemplary cells (5.0×10⁴/well) were transferred to a round-bottomed 96-well enzyme-linked immunosorbent assay (ELISA) plate and treated with exemplary CsA-free liposomes (20 μL, 100 mM), CsA-entrapping liposomes (20 μL, with 0.01 mM CsA), and free CsA (20 μL, 0.01 mM). All exemplary wells, including exemplary untreated samples, received 20 μL of phytohemagglutinin-L (PHA-L) (5 μg/ml), while exemplary control wells only received RPMI medium (0.2 mL). Following a four-day incubation period at 37° C. in a 5% CO₂ incubator, an exemplary ELISA plate was centrifuged at about 700 g for five minutes. Supernatant RPMI medium was then removed, and exemplary cell pellets in each well were resuspended in 2 mL PBS/FBS solution. Finally, fluorescence intensity of exemplary cells was measured using flow cytometry.

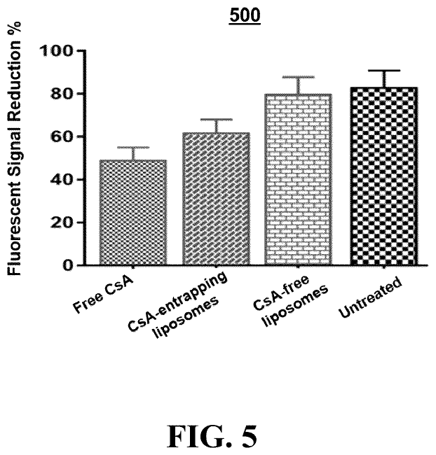

FIG. 5 illustrates bar chart 500 representing CFSE fluorescence intensity of T cells following a 4-day incubation, at 37° C. and 5% CO₂, with exemplary samples of free CsA, CsA-entrapping liposomes, and CsA-free liposomes, consistent with one or more exemplary embodiments of the present disclosure. In further detail with respect to bar chart 500, there was a significant difference in CFSE reduction between exemplary treated and untreated T cells. When compared to untreated T cells, both exemplary free CsA and exemplary CsA-entrapping liposomes effectively reduced CFSE dilution in exemplary cells. However, exemplary CsA-free liposomes were unable to reduce CFSE dilution in exemplary cells.

### Example 7: Evaluating Immunosuppressive Activity of CsA-Entrapping Liposomes Against Delayed-Type Hypersensitivity (DTH) Response

In this example, immunosuppressive effects of CsA-entrapping liposomes and free CsA were evaluated against sheep red blood cells (SRBC)-induced delayed-type hypersensitivity reaction in rats. For this purpose, an intraperitoneal (IP) injection of SRBC (1×10⁹ cells/0.2 mL injection volume) was given to all exemplary rats except for an exemplary sham/control group, which just received a PBS injection (0.2 mL/rat). Then, after two hours, CsA-entrapping liposomes and free CsA were intravenously injected at 2 mg CsA/Kg animal weight. On day six, exemplary animals were given another injection of SRBC, this time subcutaneously (SC) to left hind footpad, with the exception of an exemplary sham group which received PBS. On day six, a spherometer (pitch 0.01 mm) was used to measure thickness of left hind footpad.

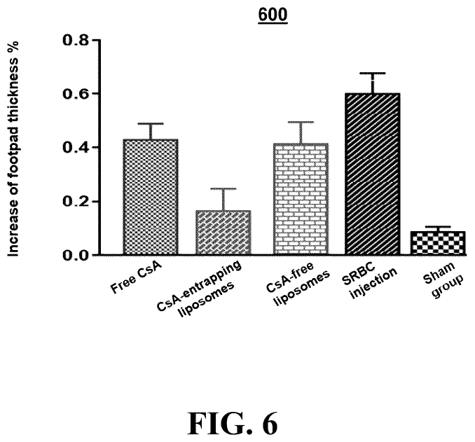

FIG. 6 illustrates bar chart 600 representing immunosuppressive activity of exemplary free CsA, CsA-entrapping liposomes, and CsA-free liposomes against an exemplary SRBC-induced DTH reaction in rats, determined by measuring inflammatory edema of rats' left hind footpad, 24 h after subcutaneous injection of SRBCs, consistent with one or more exemplary embodiments of the present disclosure. In further detail with respect to bar chart 600, it was found that both exemplary CsA-entrapping liposomes and free CsA significantly suppressed inflammatory reactions in all exemplary rats. In comparison to untreated rats, exemplary CsA-entrapping liposomes and free CsA significantly reduced edema-related footpad development. It was determined that exemplary CsA-entrapping liposomes were significantly effective treatment for edema-related footpad development. It was found that exemplary CsA-entrapping liposomes were much more effective than both exemplary free CsA and exemplary CsA-free liposomes.

Serum levels of Interleukin-2 (IL-2) were measured 14 days post-DTH induction using an ELISA kit. Blood samples were taken from a retrobulbar venous sinus and allowed to clot at room temperature for 30 minutes. An exemplary serum was collected in a tube and then diluted with the same volume of normal saline after centrifugation (2500 rpm, 15 min). An exemplary standard solution and an exemplary diluted serum (50 μL/well) were transferred to a 96-well plate containing an exemplary assay diluent (50 μL). An exemplary plate was then covered and sealed with an adhesive strip before being incubated for 2 hours at about 20-25° C. (i.e., room temperature). After washing steps (×5) with an exemplary wash buffer, Rat IL-2 (Interleukin-2) Conjugate (100 μL) was added to exemplary wells, and an exemplary plate was covered and incubated for another 2 hours. Subsequent to repeating exemplary washing step (×5) with an exemplary wash buffer, an exemplary substrate solution (100 μL) was added to each well and an exemplary plate was incubated in darkness for about 30 minutes. Finally, an exemplary stop solution (100 μL) was added, and optical density of each well was examined at 540 nm using a microplate reader.

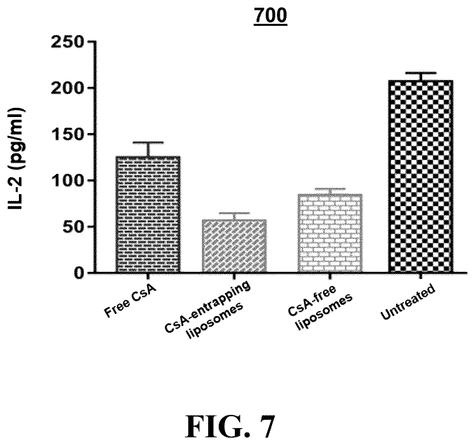

FIG. 7 illustrates bar chart 700 representing immunosuppressive activity of exemplary free CsA, CsA-entrapping liposomes, and CsA-free liposomes against an exemplary SRBC-induced DTH reaction in rats, determined by measuring an exemplary serum level of IL-2 fourteen days after subcutaneous injection of SRBC, consistent with one or more exemplary embodiments of the present disclosure. In further detail with respect to bar chart 700, it was found that both exemplary free CsA and CsA-entrapping liposomes significantly decreased serum levels of IL-2. Notably, the CsA-entrapping liposomes significantly reduced IL-2 levels compared to free CsA and CsA-free liposomes. Among all treatments, CsA-entrapping liposomes demonstrated the greatest reduction in IL-2 levels.

The levels of serum Immunoglobulin M (IgM) were assessed in rat blood samples on day 14 following induction of DTH. Initially, an exemplary rat serum was diluted 1:500 using 1× sample diluent and then transferred to a 96-well plate (50 μL/well) that already contained 20 μL of an exemplary standard solution, with concentrations ranging within 0-10 μg/mL. Additionally, 80 μL of 1× sample diluent was added to each well and gently mixed. An exemplary plate was incubated with these reagents for one hour at 20-25° C. (i.e., room temperature) before being washed three times with an exemplary wash buffer. Subsequently, 100 μL of antibody-enzyme conjugate was added to each well and gently mixed using a stirrer. After a 30-minute incubation period, wells were washed five times with an exemplary wash buffer and then treated with an exemplary substrate solution (TMP). Following a further incubation period of 15 minutes, an exemplary stop solution (100 μL) was added to each well, and the plate was allowed to incubate at 20-25° C. (i.e., room temperature) until the colour of exemplary wells transitioned from blue to yellow. Lastly, using a microplate reader and a standard curve, the concentration of IgM in an exemplary serum was determined at an absorbance wavelength of about 450 nm.

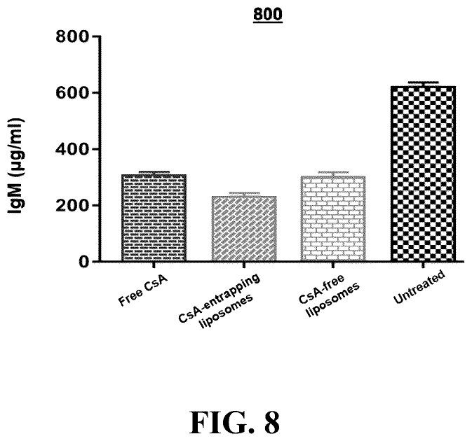

FIG. 8 illustrates bar chart 800 representing immunosuppressive activity of exemplary free CsA, CsA-entrapping liposomes, and CsA-free liposomes against an exemplary SRBC-induced DTH reaction in rats, determined by measuring serum levels of IgM fourteen days after subcutaneous injection of SRBC, consistent with one or more exemplary embodiments of the present disclosure. In further detail with respect to bar chart 800, exemplary CsA-entrapping liposomes demonstrated the lowest levels of IgM when compared to other treatments, including treatment with exemplary free CsA and CsA-free liposomes.

### Example 8: Biodistribution Profile of CsA-Entrapping Liposomes

In this study, biodistribution characteristics of exemplary CsA-entrapping liposomes were evaluated in male rats. For this purpose, an intravenous injection of both exemplary CsA-entrapping liposomes and free CsA was administered to exemplary rats at a dosage of 2 mg drug/kg. Blood samples were subsequently collected from tail vein into heparinized tubes using a sterile scalp vein set at various time intervals: 0.5, 1, 2, 4, 6, 12, 24, and 48 hours. These samples were maintained at 20° C. for further analysis. CsA concentrations in blood were determined using a chemiluminescence immunoassay analyser after extracting whole blood CsA with a cyclosporine measurement kit. Additionally, tissue CsA concentrations were assessed through a chemiluminescence immunoassay (CLIA) conducted 48 hours post-injection of exemplary liposomes.

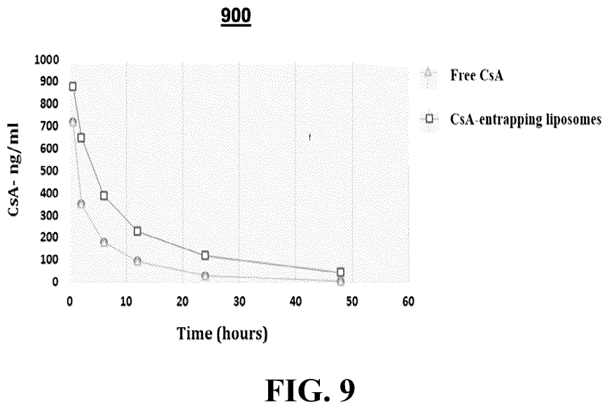

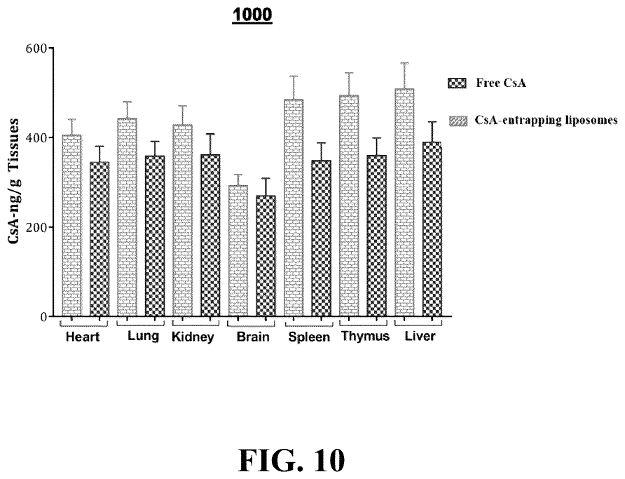

FIG. 9 illustrates bar chart 900 representing biodistribution profile of both exemplary free CsA and CsA-entrapping liposomes, determined by measuring serum level of CsA at various time points following intravenous injection of exemplary CsA-entrapping liposomes and free CsA in rats, consistent with one or more exemplary embodiments of the present disclosure. FIG. 10 illustrates bar chart 1000 representing biodistribution profile of both exemplary free CsA and CsA-entrapping liposomes, determined by measuring levels of extracted CsA from various tissues 48 h after intravenous administration of exemplary CsA-entrapping liposomes and free CsA in rats, consistent with one or more exemplary embodiments of the present disclosure. As shown in bar chart 900, the concentration of CsA in blood and tissues significantly increased over time when treating with exemplary CsA-entrapping liposomes. Table 4 below presents the pharmacokinetics of exemplary free CsA and CsA-entrapping liposomes after intravenous injection into male rats.

TABLE 4
Pharmacokinetics of exemplary free CsA and CsA-entrapping liposomes
after intravenous injection into male rats, consistent with one
or more exemplary embodiments of the present disclosure.

| Parameter | Free-CsA | CsA-Entrapping liposomes |
|---|---|---|
| T_{1/2} (hour) | 8.6 | 15.5 |
| Cₘₐₓ (ng/ml) | 720 | 880 |
| AUC₀₋ₜ (ng/ml*h) | 4271 | 9648 |
| Clearance ((mg/kg)/(ng/ml) *h) | 2.3 × 10⁻⁴ | 0.9 × 10⁻⁴ |

In further detail with respect to bar chart 1000, exemplary CsA-entrapped liposomes were found to enhance the concentration of CsA in certain tissues 48 hours after injection. An increase in CsA levels was observed in lungs, kidneys, spleen, thymus, and liver of an exemplary group treated with exemplary CsA-entrapping liposomes compared to exemplary tissue samples from an exemplary group treated with an exemplary free CsA. The highest concentrations of CsA were detected in spleen, thymus, and liver, where exemplary CsA-entrapping liposomes demonstrated the highest levels of CsA within these tissues. As a result of CsA increase, exemplary pharmacokinetic parameters associated with exemplary CsA-entrapping liposomes were significantly different from those of exemplary free CsA. For example, half-life (T_{1/2}) of CsA increased dramatically from 8.6 hours in rats that were treated with exemplary free CsA to about 15.5 hours in rats treated with exemplary CsA-entrapping liposomes. The area under the curve (AUC₀₋ₜ) of CsA blood profile was approximately doubled in exemplary CsA-entrapping liposomes (9648 ng/mL*h CsA in rats treated with exemplary CsA-entrapping liposomes compared to 4271 ng/mL*h CsA in rats treated with exemplary free CsA). Meanwhile, clearance (Cl) of CsA from rats blood was halved (2.3×10⁻⁴ (mg/kg)/(ng/ml)*h in rats treated with exemplary free CsA compared to 0.9×10⁻⁴ (mg/kg)/(ng/ml)*h in rats treated with exemplary CsA-entrapping liposomes), however the peak concentration (Cₘₐₓ) of CsA in blood was similar in groups (i.e., rats treated with exemplary CsA-entrapping liposomes and rats treated with exemplary free CsA).

In regards to tissue distribution, the use of exemplary CsA-entrapping liposomes significantly enhanced the concentration of CsA in certain tissues 48 hours post-injection. The amounts of CsA in lung, kidney, spleen, thymus, and liver were significantly higher in exemplary rats treated with exemplary CsA-entrapping liposomes compared to exemplary treated with free CsA. Spleen, thymus, and liver exhibited the highest concentrations of CsA, with the highest levels observed in exemplary rats administered with exemplary CsA-entrapping liposomes within these specific tissues.

### Example 9: Evaluating Skin Allograft Rejection Following Injection of CsA-Entrapping Liposomes

In this example, skin allograft rejection was evaluated in response to a single-dose injection of exemplary CsA-encapsulated liposomes, CsA-free liposomes, and free CsA. To do this, exemplary male mice were given an intravenous injection of exemplary CsA-entrapping liposomes and free CsA at a dose of 2 mg/kg. Exemplary mice were then anesthetized using a ketamine/xylazine mixture 24 hours post-injection. An exemplary tail skin of each mouse was carefully removed with a sterile scalpel and transplanted onto the back of the same mouse. On days 7, 12, 17, and 22 post-transplantation, an exemplary graft area was dressed and inspected for signs of skin rejection and histological changes. At these time points, an exemplary grafted skin area was excised from exemplary host mice using a scalpel and preserved in a 10% neutral buffered formalin solution.

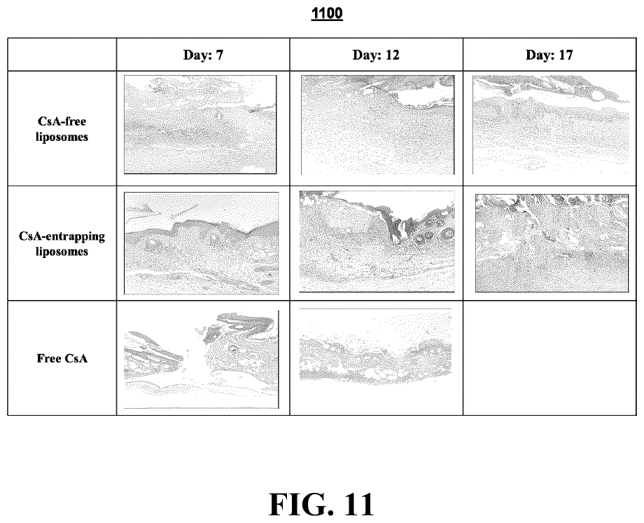

After 24 hours, exemplary tissue samples were embedded in paraffin and sectioned to a thickness of about 5 mm. Exemplary sections were then stained with hematoxylin and eosin (H&E). Lastly, an independent reviewer who was blinded to exemplary treatment groups examined exemplary histological slides under light microscopy for any indications of rejection such as inflammatory cell infiltration, fibroplasia formation, epithelialization, or necrosis. FIG. 11 illustrates microscopic images 1100 representing histopathological features of exemplary transplanted skin tissues in exemplary host mice treated with a single dose (2 mg/mL) of exemplary free CsA, CsA-entrapping liposomes, and CsA-free liposomes, consistent with one or more exemplary embodiments of the present disclosure. In further detail with respect to microscopic images 1100, skin allograft rejection was significantly delayed following a single dose (2 mg CsA/kg weight) injection of CsA-entrapping liposomes. Compared to free CsA and CsA-free liposomes, the onset of pathogenic symptoms was slowed. Pathological features, such as development of newly connective tissues (NCT) between graft and host skin, epithelialization of grafted tissue, infiltration of inflammatory cells into host skin, loss of epidermal layer, and skin rejection due to development of necrotic areas in grafted tissues were observed.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for producing liposomes entrapping Cyclosporine A (CsA), the method comprising:
preparing a suspension of CsA-free liposomes in a sucrose phosphate buffer, wherein the suspension of CsA-free liposomes in the sucrose phosphate buffer has a pH level of 7 and comprises CsA-free liposomes with a concentration of 50 mM by volume of the suspension of CsA-free liposomes in the sucrose phosphate buffer, wherein preparing the suspension of CsA-free liposomes in the sucrose phosphate buffer comprises of:
preparing a chloroform-lipid solution by dissolving dioleoyl phosphatidylethanolamine, dioleoyl phosphatidylserine, 1,2-distearoyl-sn-glycero-3-phosphoethanolamine-N-[amino(polyethyleneglycol)-2000], cholesterol, and alpha tocopherol in chloroform, wherein the chloroform-lipid solution comprises 17.5 mM dioleoyl phosphatidylethanolamine, 20 mM dioleoyl phosphatidylserine, 2.5 mM 1,2-distearoyl-sn-glycero-3-phosphoethanolamine-N-[amino(polyethyleneglycol)-2000], 9.75 mM cholesterol, and 0.25 mM alpha tocopherol;
forming a lipid film by evaporating the chloroform from the chloroform-lipid solution;
preparing a suspension of vesicles by hydrating the lipid film with a mannitol acetate buffer comprising:
preparing a mixture of the lipid film and the mannitol acetate buffer by adding the mannitol acetate buffer with a pH level of 7 and a temperature level of 45° C. to the lipid film, wherein the mannitol acetate buffer has a mannitol concentration of 300 mM by volume of the mannitol acetate buffer; and
forming the suspension of vesicles by agitating the mixture of the lipid film and the mannitol acetate buffer while a temperature level of the mixture of the lipid film with the mannitol acetate buffer is maintained at 45° C.;
extruding the suspension of vesicles through a polycarbonate membrane with a pore size of 200 nm, a polycarbonate membrane with a pore size of 100 nm, and a polycarbonate membrane with a pore size of 50 nm, respectively; and
forming the suspension of CsA-free liposomes in the sucrose phosphate buffer by dialyzing the extruded suspension of vesicles against a sucrose phosphate buffer with a pH level of 7 and a sucrose concentration of 280 mM;
preparing a mixture with a pH level of 7 by adding the suspension of CsA-free liposomes in the sucrose phosphate buffer to an ethanolic solution of CsA with a CsA concentration of 83 mM; and
forming the liposomes entrapping CsA by incubating the mixture at a temperature level of 45° C. for a time duration of 60 minutes.

2. A method for producing liposomes entrapping Cyclosporine A (CsA), the method comprising:
preparing a suspension of CsA-free liposomes in a sucrose phosphate buffer, wherein the suspension of CsA-free liposomes in the sucrose phosphate buffer has a pH level of 7 and comprises CsA-free liposomes with a concentration between 45 mM and 55 mM by volume of the suspension of CsA-free liposomes in the sucrose phosphate buffer, wherein preparing the suspension of CsA-free liposomes in the sucrose phosphate buffer comprises of:
preparing a lipid solution by dissolving a plurality of lipids and alpha tocopherol in chloroform;
forming a lipid film by evaporating the chloroform from the lipid solution;
preparing a suspension of vesicles by hydrating the lipid film with a mannitol acetate buffer comprising:
preparing a mixture of the lipid film and the mannitol acetate buffer by adding the mannitol acetate buffer with a pH level of 7 and a temperature level between 42° C. and 48° C. to the lipid film, wherein the mannitol acetate buffer has a mannitol concentration between 295 mM and 310 mM by volume of the mannitol acetate buffer; and
forming the suspension of vesicles by agitating the mixture of the lipid film and the mannitol acetate buffer while a temperature level of the mixture of the lipid film with the mannitol acetate buffer is maintained between 42° C. and 48° C.;
extruding the suspension of vesicles through a polycarbonate membrane with a predetermined pore size; and
forming the suspension of CsA-free liposomes in the sucrose phosphate buffer by dialyzing the extruded suspension of vesicles against a sucrose phosphate buffer with a pH level of 7 and a sucrose concentration between 270 mM and 290 mM;
preparing a mixture with a pH level of 7 by adding the suspension of CsA-free liposomes in the sucrose phosphate buffer to an ethanolic solution of CsA with a CsA concentration between 70 mM and 90 mM; and
forming the liposomes entrapping CsA by incubating the mixture at a temperature level between 42° C. and 48° C. for a time duration between 55 and 65 minutes.

3. The method of claim 2, wherein the plurality of lipids comprises:
a plurality of phospholipids comprising at least one of dioleoyl phosphatidylethanolamine, dioleoyl phosphatidylserine, and a polyethylene glycol (PEG)-modified phospholipid; and
a plurality of cholesterol molecules dispersed among the plurality of phospholipids.

4. The method of claim 3, wherein the PEG-modified phospholipid is a 1,2-Distearoyl-sn-glycero-3-phosphoethanolamine-PEG conjugate.

5. The method of claim 2, wherein the suspension of CsA-free liposomes in the sucrose phosphate buffer comprises CsA-free liposomes with a concentration of 50 mM by volume of the suspension of CsA-free liposomes in the sucrose phosphate buffer.

6. The method of claim 2, wherein the mannitol acetate buffer has a mannitol concentration of 300 mM by volume of the mannitol acetate buffer.

7. The method of claim 2, wherein the ethanolic solution of CsA has a CsA concentration of 83 mM.

8. The method of claim 2, wherein extruding the suspension of vesicles through the polycarbonate membrane with the predetermined pore size comprises:
extruding the suspension of vesicles through a polycarbonate membrane with a pore size of 200 nm, a polycarbonate membrane with a pore size of 100 nm, and a polycarbonate membrane with a pore size of 50 nm, respectively.

9. The method of claim 2, wherein dialyzing the extruded suspension of vesicles against the sucrose phosphate buffer with the pH level of 7 and the sucrose concentration between 270 mM and 290 mM comprise dialyzing the extruded suspension of vesicles against the sucrose phosphate buffer with the pH level of 7 and a sucrose concentration of 280 mM.
